(19) United States
(12) Patent Application Publication
(10) Pub. No.: US 2010/0184123 A1
(43) Pub. Date: Jul. 22, 2010

(54) Gas Exchange Detection Method and Device

(75) Inventors: John Martyn Fall, Essex (GB); Jan Lowery Groves, Essex (GB); Elizabeth Ann Howlett Hall, Hampshire (GB); James Ormiston Rutherford, Cambridge (GB)

Correspondence Address: PETERS VERNY , L.L.P., 425 SHERMAN AVENUE, SUITE 230, PALO ALTO, CA, 94306, US

(73) Assignee: METRICA.BIO LIMITED, Halstead (UK)

(21) Appl. No.: 12/376,696

(22) PCT Filed: Aug. 7, 2007
(86) PCT No.: PCT/GB07/03002

§ 371 Date: May 14, 2009

(30) Foreign Application Priority Data
Aug. 8, 2006 (GB) 0615678.0

(51) Int. Cl. C12M 1/34; C12Q 1/04

(52) U.S. Cl. 435/34; 435/287.5

(57) ABSTRACT

The invention relates to a gas exchange detection method and device particularly, but not exclusively, for rapid detection of the presence of micro-organisms, the effect of an additional substance upon a micro-organism and/or the rate at which oxygen is consumed, in solutions, aqueous emulsions and/or suspensions.

The invention relates to a gas exchange detection method and device particularly, but not exclusively, for rapid detection of the presence of micro-organisms, the effect of an additional substance upon a micro-organism and/or the rate at which oxygen is consumed, in solutions, aqueous emulsions and/or suspensions.

In many fields, it is important to be able to determine whether materials are contaminated with micro-organisms. For example, in the medical field, micro-organisms can cause serious infection, which can be life threatening if not detected in a timely fashion. Furthermore, in a number of industrial areas, such as the food, cosmetic and sewage industries, detection of microbiological contamination or toxicity is of the utmost importance.

Whilst there are a number of methods that can be used to detect micro-organisms, for example colony count enumeration, bioluminescence and impedimetry, recent studies suggest that short-term respirometry is an easier, more rapid and reliable methodology (M Simões et al.: Biofouling, 2005; 21(1): 9-17).

Although, a number of techniques are presently available for measuring gas production of micro-organisms, each has a number of disadvantages or limitations.

EP 0611821 (Commissariat a L'Energie Atomique) describes a process and device that determines biological activity by growing two cultures of micro-organisms in separate containers and comparing the pressure difference between the containers after a suitable length of time. However, the pressures are measured independently and the pressure within the first container must reach a pre-determined threshold before measurement of the pressure difference can be achieved. Sensitivity is also described as being as high as 25×10² Pa.

WO 03/078563 (Bactest Limited) describes an indirect pressure sensing system for non-invasive monitoring of gas exchange by biological material. However, this arrangement provides indirect pressure measurement, for example, pressure in a first container is measured by sensing pressure in a second container, which is sealed and isolated from the first container by a flexible diaphragm.

It is therefore an object of the invention to provide a method and device which achieves a rapid measurement of microbiological activity and is sufficiently sensitive to detect low levels of microbiological activity without the need for a cultivating or concentration step.

Thus, according to a first aspect of the invention, there is provided a method for determining the gas exchange of a test sample which comprises the steps of:
(a) adding a reference sample to a reference chamber;
(b) adding the test sample to a test chamber;
(c) sealing the chambers from the external environment; and
(d) directly measuring the pressure differential between each of said chambers over a specific time period.

The method of the invention is capable of monitoring gas uptake, evolution or exchange of a sample comprising biological or chemical components at extremely low levels by continuously monitoring the pressure difference between the reference chamber and test chamber and the rate at which it occurs. The increased sensitivity removes the need for a cultivating or concentration step, which helps to speed up the preparation of the test, and enables the method to achieve detection nearly 600 times faster than traditional microbiological culturing.

Furthermore, capturing multiple measurements of pressure offers numerous advantages when compared with taking one measurement. For example, the invention can provide the user with significantly more information with respect to the sample's components, such as the enzyme-substrate reaction kinetics, the time required to modify a bacteria culture's respiration, the uptake of oxygen prior to release of CO₂, inhibition or enhancement of respiration following addition of biocide, antibiotic or nutrient etc. In one embodiment, the pressure differential is measured once per second over the duration of the specific time period.

It will also be appreciated that the simultaneous use of the reference chamber containing a reference (or control sample) having a similar matrix to the test sample helps to eliminate any errors resulting from variations in conditions that may occur if the samples were analysed separately.

In one embodiment, each chamber is partially filled with a sample comprising components capable of gas exchange and, therefore, of producing a pressure variation in the chamber's headspace. The components may be biological, e.g. respiring bacteria, or non-biological, such as an organic or inorganic chemical, e.g. an enzyme or volatile substance. Where the components are biological, pressure changes are a consequence of oxygen depletion caused by the respiring living organisms in the sample or release of a gas such as, but not exclusively carbon dioxide.

In one embodiment, the test sample is a sample with an unknown level of sterility or micro-organism activity, whilst the reference sample contains a reference (or control) sample, having a similar matrix to the test sample, but with a known level of sterility or micro-organism activity. In one embodiment, the reference sample may be free of micro-organisms (e.g. the sample may be inactive or sterile).

In an alternative embodiment, the test and reference samples each have a known level of sterility or micro-organism activity. In a further embodiment, the test and reference samples have equivalent levels of sterility or micro-organism activity (e.g. the levels of activity vary only from 1-5%). In a further embodiment, the test and reference samples have identical levels of sterility or micro-organism activity. Such an embodiment provides the advantage of allowing the process to monitor biocide or nutrient efficacy upon the test sample while using the reference sample for comparison.

In one embodiment, each sample is introduced into the chamber in a receptacle.

Pre-treatment of samples is usually un-necessary in steps (a) and (b), unless activity levels are so high that they are outside the pressure measuring range of the device. If this is the case, then aqueous dilution is required to bring the sample within the measuring range of the device. The sealing step (c) is necessary to achieve gas-tight chambers and is generally achieved by providing sealing means (e.g. a lid and o-ring) over each of the chambers.

In one embodiment, one or more parameter adjustment steps are conducted prior to pressure measurement in step (d).

In one embodiment, the parameter adjustment step comprises temperature adjustment. The activity of any given component (e.g. a micro-organism) will typically vary with temperature and each component typically has an optimum temperature. In this embodiment, the parameter adjustment step may require a pre-heating step to an optimum and/or constant temperature. An incubation step may also be required to enable the components of the sample to move from semi-dormancy to a more active state once an optimum temperature has been reached. The incubation step may also assist with removal of dissolved gases from the solution, which may mask any pressure change due to bacterial respiration. In one embodiment, the incubation step has a duration of between 1 and 6000 seconds, suitably between 10 and 600 seconds.

In one embodiment, the parameter adjustment step comprises pressure adjustment. In this embodiment, the pressure sensor is zeroed by opening exhaust valves and equalising the pressure in each chamber. In this embodiment, the parameter adjustment step may require a venting step to allow equilibration of the internal pressure of the chamber. In one embodiment, the venting step has a duration of between 5 and 60 seconds.

In one embodiment, the parameter adjustment step comprises mixing adjustment. For example, generally the rate and length of mixing affects gas exchange. In one embodiment, mixing occurs throughout pressure measurement step (d). In an alternative embodiment, mixing occurs intermittently.

In one embodiment, the parameter adjustment step may be conducted by processing means as defined herein (e.g. use of a set-up menu). Once optimised for a particular component, the parameters can be stored as an automatic setting. A menu-based system allows for the optimal setting of conditions for a wide variety of samples.

Once the parameters have been adjusted, the relative pressure change differences between the two chambers in step (d) is measured. Numerous measurements are made, such as 1/second, over a specific measuring period.

In a further embodiment, venting, temperature and/or mixing are controlled by processing means during the test to ensure that these parameters remain constant. For example, if the temperature is raised above or lowered below the initial starting temperature during the test, then the processing means is configured to adjust the temperature accordingly by decreasing or increasing power to the heating mat respectively.

It will be appreciated that different components will differ in their activity and consequently the length of the measuring period can be varied in order to take account of these discrepancies. For example, samples with low levels of activity may need a longer cycle time to ensure that small pressure changes can be clearly distinguished. In one embodiment, the measuring cycle can be varied between 30 and 6000, typically between 30 and 600 seconds using the processing means.

In one embodiment, step (d) is repeated one or more times. In a further embodiment, step (d) is repeated with identical samples (e.g. samples having the same concentration). In a yet further embodiment, step (d) is repeated with differing samples (e.g. samples having different concentrations). This embodiment provides the advantage of allowing the study of enzyme-substrate reaction kinetics (e.g. a concentration dependent analysis of enzyme-substrate reactions).

The pressure measurements may be transferred to processing means and processed simultaneously with step (d). In one embodiment, the processing means are arranged to display pressure changes numerically during a test. This numeric data can then be relayed to a computer, where manipulation allows the presentation of results in a graphical format.

In a further embodiment, the processing means is capable of full data processing, i.e. it can calculate the rate of change in pressure. In a yet further embodiment, the processing means additionally displays the real-time, pressure changes in a graphical format during the test. In a yet further embodiment, the display may also show the status of various conditions, e.g. pressure, temperature and mixing.

A result is available within a few minutes, depending upon the parameters set for the run. The user has the ability to calculate gradient of the pressure change from a selected part of the plot. The system also allows archived plots to be overlaid onto the result. The data can then be manipulated, labelled and/or time stamped.

According to a further aspect of the invention, there is provided a pressure sensing device comprising a plurality of sealable chambers and a pressure sensor which is configured, in use, to measure multiple pressure differences between any of said chambers over a specific time period.

According to a second aspect of the invention, there is provided a pressure sensing device comprising a plurality of sealable chambers and a direct pressure differential measurement sensor which is configured, in use, to directly measure the pressure differential between any of said chambers over a specific time period.

In one embodiment, the plurality of sealable chambers comprise one or more test chambers and a reference chamber. In a further embodiment, the plurality of sealable chambers comprise a test chamber and a reference chamber.

In one embodiment, each chamber comprises a removable receptacle. This embodiment allows the sample to be conveniently introduced and contained within the chamber during analysis. Such an arrangement provides a significant advantage of allowing sample analysis on a large scale. For example, multiple samples may be obtained and prepared simultaneously. Such samples may then either be analysed immediately in the device, or they may be stored and subsequently analysed by the device in a laboratory. The presence of sterile, single-use, disposable receptacles further eliminates the need for cleaning and/or sterilising each chamber between analyses. For example, after each analysis the receptacle can be discarded and a further receptacle can immediately be placed into the chamber. Such an embodiment allows the device to be either a laboratory based unit or a remote portable unit. It will also be appreciated that stirrers may also be disposable for convenience.

In a further embodiment, the receptacle is constructed of a rigid material, suitably glass, or a mouldable or formable plastic.

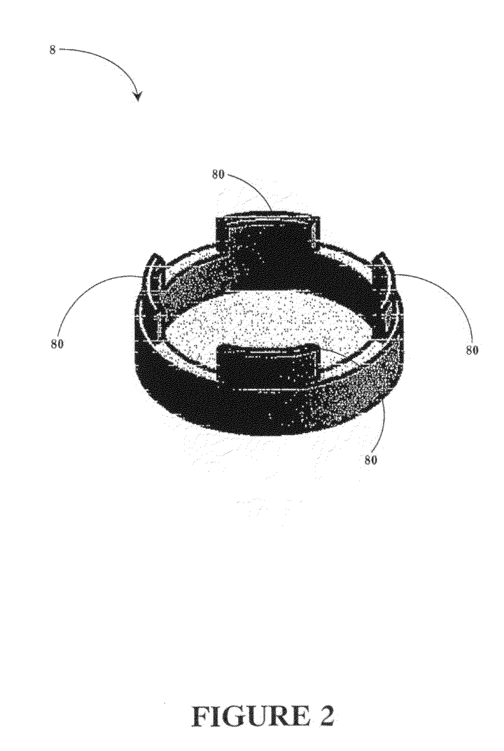

As the invention is based on gas exchange, it is desirable to ensure that there is adequate interaction between the liquid and gas phases. For this reason, the size and shape of the chamber and receptacle should be selected to allow optimum conditions for gas exchange. For example, the receptacle may be moulded substantially as a hollow cylinder, with a flat base and an open top. In one embodiment, the top may comprise discrete vertical, upwardly extending protrusions to facilitate easier manipulation of the receptacle. In a further embodiment, the receptacle is shaped substantially as illustrated in FIG. 2. It will be appreciated that the walls of the receptacle will be arranged so that they are immediately adjacent to the walls of the chamber.

In one embodiment, each chamber comprises mixing means. Gentle stirring provides the advantage of facilitating oxygen exchange between the liquid and gas phase. In one embodiment, the mixing means comprises one or more magnetic stirrers, such as those known in the art. Each magnetic stirrer typically comprises a magnet rotated by a motor located outside the chamber and a metallic stirrer placed within the receptacle in the chamber. In one embodiment, the motor is a DC motor.

The DC motors and magnets are configured to create a rotating magnetic field when each DC motor is activated, which causes the magnetic stirrers to rotate at exactly the same rate and stir the samples within the chamber. Since the magnetic coupling operates through the wall of the chamber and receptacle, there are no seals, apertures or other leakage points. Furthermore, there is no need for lubricants, which can contaminate the sample and its components. The speed at which the motors and the magnetic stirrers rotate can be varied.

In one embodiment, the volume of the receptacle is in the range of 500 μl to 7000 μl, suitably 3000 μl to 4000 μl where 500 to 1500 μl is the liquid (i.e. sample) phase and 4000 to 2000 μl is headspace atmosphere. In a further embodiment, the volume of the receptacle is 3400 μl, wherein 1000 μl is the liquid (i.e. sample) phase and 2400 μl is headspace atmosphere.

In a further embodiment, the receptacle comprises a headspace marker (e.g. a mark, line, groove or the like) to define the volume of sample required to give the optimum ratio of headspace to sample ratio. In a yet further embodiment, when the volume of the receptacle is 3400 μl, the headspace marker is present at 1000 μl.

Where the sample comprises biological components, the organisms will expire CO, and offset some of the pressure drop due to oxygen depletion.

Therefore in one embodiment, each chamber additionally comprises a suitable gas (e.g. CO₂) absorbing material to remove specific volatile compounds. In one embodiment, the CO₂ absorber is soda lime. In a further embodiment, the CO₂ absorber is present in the chamber headspace.

In one embodiment, the chambers are sealed by one or more closure means. In a further embodiment, the closure means comprise a lid. The chambers are designed to be gas tight when the lid is closed. It will be appreciated that in this embodiment, the chambers will be sealed from both the atmosphere and each other.

In one embodiment, each chamber additionally comprises exhaust means. This embodiment allows communication of the chamber's environment with the external environment. In a further embodiment, the exhaust means comprise a gas-tight valve and connecting means from the valve to the chamber. In a yet further embodiment, the valve is an on-off solenoid valve, which is well known to those skilled in the art. In an alternative embodiment, the valve is a 3 way solenoid valve. Equilibration of the chamber pressure with ambient pressure can be carried out by opening the valves.

In one embodiment, the device comprises means for connecting each chamber to the pressure sensor. In a further embodiment, the connecting means comprise gas-tight tubes. The port sizes and tubing configurations of the connecting means are important variables that have been optimised. For example, the tubing may have a thick wall, a small diameter and a short overall length to minimise its contribution to the headspace volume. In one embodiment, the tubing has a bore between 1 to 2 mm, suitably 1.6 mm, and an outside diameter between 4 to 6 mm, e.g. 4.8 mm.

The direct pressure differential measurement sensor may be of any type suitable for detecting small pressure variations from about 0.1 Pa to 200 Pa. It may also be capable of providing positive or negative pressure changes relative to the reference chamber. The device will typically comprise an integrated circuit transducer which converts pressure into a voltage signal output. In one embodiment, the pressure sensor is an amplified pressure sensor (e.g. any pressure sensor which can be obtained from SENSORTECHNICS GmbH, Boschstr. 10, 82178 Puchheim, Germany, such as 113LP01D, 113LP02D or 113LP05D).

In one embodiment, the pressure sensor is linked either directly or indirectly to processing means arranged to output the pressure differences between any of said chambers.

As used herein, the term ‘processing means’ refers to a computer, for example any conventional PC or Mac, desktop, laptop or pocket personal computer or PDA, or any other device capable of processing algorithms.

It will be appreciated that references to ‘linked’ include all known connections from processing means to peripheral hardware and include both wired and wireless connections. Such connections may therefore include universal serial bus (USB) port connections, IEEE 1394 external bus standard connections (also known as firewire, i.link and lynx), Bluetooth connections, serial connections, InfraRed (iRDA) connections or WIFI (IEEE 802.11).

In one embodiment, the analogue electrical output is transferred from the pressure sensor, and then relayed to the processing means. The data can be analysed to calculate the rates and significances of changes. In one embodiment, the processing means displays pressure changes numerically during a test. This numeric data can then be relayed to a computer, where manipulation allows the presentation of results in a graphical format.

In a further embodiment, the processing means is capable of full data processing, i.e. it can calculate the rate of change in pressure. In a yet further embodiment, the processing means additionally comprise a display. This allows a user to see a real-time, graphical trend in pressure change during the test. The user then has the ability to calculate gradient of the pressure change from a selected part of the plot. The system also allows archived plots to be overlaid onto the result. The graph and the numeric slope may be printed using printing means. In a further embodiment, the result may be a number, which may be ideal for pass/fail situations in a quality control test.

In a yet further embodiment, the processing means additionally comprise a real-time clock that allows results to be time and date stamped. Time/date may be displayed in a format chosen by the user. A full QWERTY keyboard also allows all samples to be readily labelled using either letters or numbers or a combination thereof. The display may also show the status of various conditions, e.g. pressure, temperature and mixing.

It will be appreciated that the device can take many forms, but should maintain precise control of temperature and control mixing effectively.

In one embodiment, the device additionally comprises temperature control means. This embodiment stabilises the chambers at a particular temperature, and excludes pressure variations due to temperature fluctuations. In a further embodiment, the temperature control means comprise a thermostatic enclosure, which surrounds the chambers, wherein heat transfer occurs mostly by thermal conduction between the wall of the chamber and the receptacle. In one embodiment, the thermostatic enclosure is a thermal block comprising aluminium and a heating mat.

Thus, the test and reference chambers may be located in a thermal block, the temperature of which can be closely controlled (+/−0.01° C.) using a heating mat or by other means, such as a Peltier element. In a yet further embodiment, the temperature can be varied, for example from 4° C. to 90° C., suitably 15° C. to 60° C., to suit the characteristics of organisms of interest before the test is run. Generally, temperature will remain constant during testing.

In a yet further embodiment, the processing means will be arranged to monitor changes in conditions, deciding the significance of these events and altering them accordingly. The processing means is therefore able to control pressure venting via the exhaust means, temperature control means mixing means and/or the duration of each stage.

The invention may utilise one or more power sources, e.g. a DC power source or an AC power source. Examples of DC power sources include cells, batteries or any regulated power supply. The electronics of the pressure measuring device is suitably battery operated to allow for portable testing without the need for an electric mains supply. The advantage of connecting the device to processing means via an IEEE 1394 or USB port or other powered connection is that the device may utilise the power supply of the processing device rather than require a separate power source.

In view of the enhanced sensitivities of the device and the speed at which results can be obtained, it will be appreciated that the invention can be applied in a number of ways. For example, the device can either be used to detect the presence of micro-organisms within any given sample, whether in a solid phase, such as a culture gel, or liquid phase, or it may be used to monitor activity levels of a specific species, a chemical or, indeed, a complex mixture. Furthermore, the device can examine the effect of an additional substance upon a micro-organism to assess whether it enhances or inhibits activity. In this way, potential nutrients, preservatives, pollutants or antibiotics can be examined for any influence on micro-organism activity levels. Finally, the device can also detect the rate at which oxygen is consumed, in solutions, aqueous emulsions and/or suspensions. These tests can be carried out on raw materials, in-process products, finished products, and the plant environment.

These applications can be used in a variety of fields including, but not limited to, the medical field, environmental field, domestic applications and a number of related industries. For example, the device is capable of testing for bacterial activity in biomedical applications. For example, the device may find particular use in hospitals and surgeries which can be easily and regularly analysed for rapid assessment and treatment of bacterial infection.

Furthermore, the invention can also be used to determine microbial activity in natural and synthetic latex, cosmetic and paint samples. Contamination is a critical quality issue for manufacturers in these industries and control using existing technologies carries significant cost as well as causing delay during the manufacturing process. Applications also exist in the food industry where contamination is a major safety concern.

Additionally, the device can also be used to monitor biocide efficacy in the control of bacterial or fungal growth in water treatment applications or in any aqueous emulsion and monitor potential toxicity of incoming effluents in water treatment plants.

Thus, according to a third aspect of the invention, there is provided a use of the device as hereinbefore defined in any of the fields or industries as described hereinbefore.

In a further embodiment, there is provided a use of the device as hereinbefore defined in monitoring biocide efficacy.

According to a further aspect of the invention, there is provided a micro-organism detection kit which comprises a device as defined hereinbefore and instructions to use said kit in accordance with the process as hereinbefore defined.

In one embodiment, the kit additionally comprises one or more removable receptacles.

The invention will now be described, by way of example only, with reference to the accompanying drawings in which:

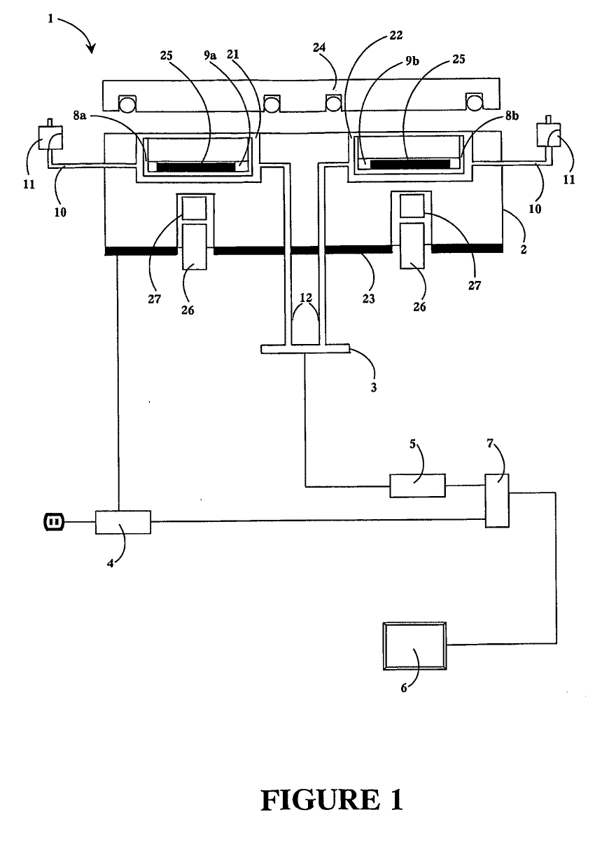

FIG. 1 shows a schematic view of the pressure sensing device according to one embodiment of the invention;

FIG. 2 shows a schematic view of the removable receptacle according to one embodiment of the invention;

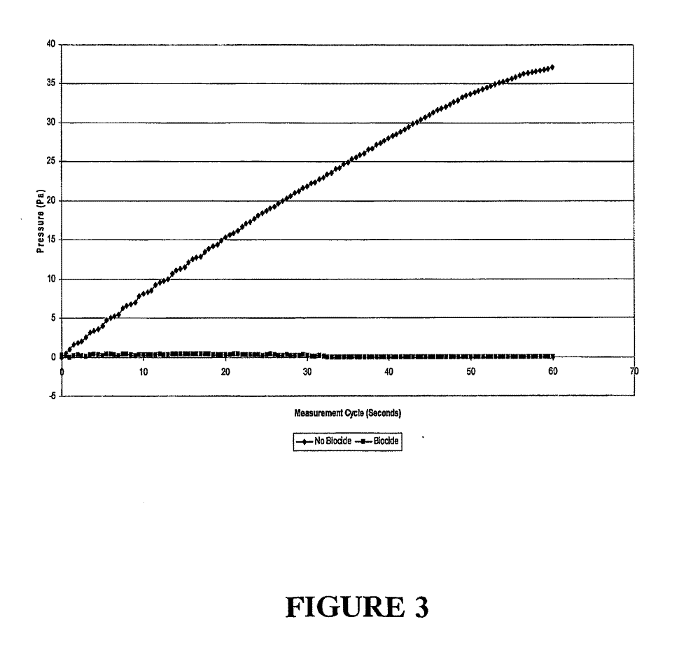

FIG. 3 demonstrates the results of an analysis of biocide efficacy in the control of bacterial growth in nitrile latex; and

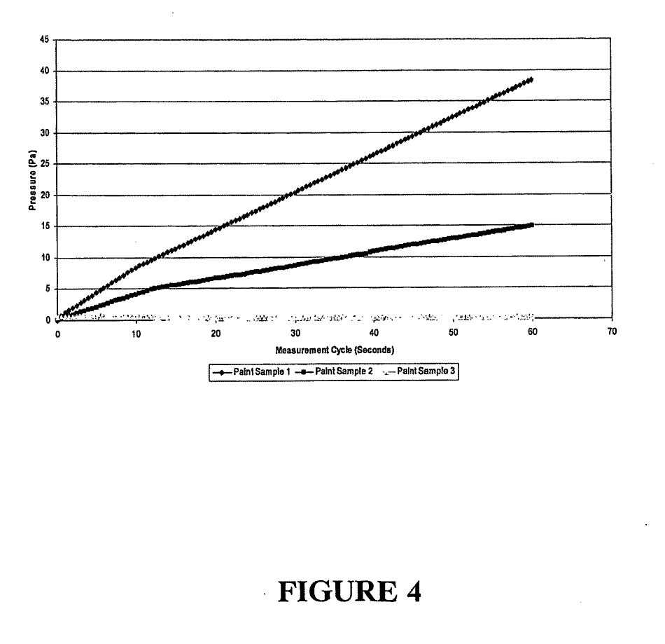

FIG. 4 demonstrates the results of an analysis of microbial activity in different paint samples.

In FIG. 1, a pressure sensing device, shown generally as 1, comprises a body 2, a pressure sensor 3, temperature adjusting means 4, analogue to digital signal conversion means 5, processing means 6 and an interface 7 between the device 1 and the processing means 6.

The body 2 is an aluminium thermal block which comprises two sealable chambers: a reference chamber 21 and a test chamber 22. In addition, the body 2 also comprises a heating mat 23, a sealing lid 24a magnetic stirrer bar 25, a DC motor 26 and a magnet 27.

The DC motor 26 and magnet 27 are configured to create a rotating magnetic field when the motor 26 is activated. The rotating magnetic field therefore causes the magnetic stirrer bar 25 to revolve.

Each sealable chamber, 21 and 22, comprises a receptacle 8a and 8b, respectively, that holds a sample (e.g. reference sample 9a and test sample 9b, respectively) and a magnetic stirrer bar 25. FIG. 2 shows one schematic representation of a receptacle, shown generally as 8 having cylindrical walls, a flat base and an open top. The receptacle 8 also comprises discrete vertical, upwardly extending protrusions 80 which facilitate easier manipulation of the receptacle 8.

A cylindrical gas-tight tube 10 connects each chamber 21, 22 to a solenoid valve 11, and a second gastight tube 12, connects each chamber 21, 22 to the pressure sensor 3.

The pressure sensor 3 is linked to the processing means 6 through the signal conversion means 5. The processing means 6 is also linked to the interface 7, temperature adjusting means 4, the solenoid valves 11 and the magnetic stirrers 25. In one embodiment (not shown), the interface 7 comprises a QWERTY keyboard and a display.

In use, a receptacle 8a containing a reference sample 9a is placed into the reference chamber 21, whilst a receptacle 8b containing an equal amount of test sample 9b is placed in the test chamber 22. The lid 24 is then closed and the parameters are adjusted using the processing means 6. For example, both chambers can be vented to equilibrate pressure and zero the pressure sensor 3; temperature can be set using the temperature adjusting means 4, which uniformly heats each chamber by controlling the heating mat 23 and thermal block 2; and stirring can be initiated by rotating the magnetic stirrer 25 at a uniform speed. Each sample 9a, 9b is exposed to exactly the same conditions.

The pressure sensor 3 calculates the relative pressure change between the two chambers 21, 22. Multiple measurements are made, such as 1/second, over a specific period and the resulting data is fed to the processing means 6 after being converted into a digital signal by the signal conversion means 5.

The processing means 3 can then display the real-time, pressure changes in a graphical format during analysis on the interface 7. The processing means 3 also controls the solenoid valves 11, temperature adjusting 4 means and/or the magnetic stirrers 25 during analysis to ensure that the parameters remain constant. For example, if the temperature is raised above or lowered below the initial starting temperature during analysis, then the processing means 3 adjusts the temperature accordingly by decreasing or increasing power to the heating mat 23 respectively.

A result is available within a few minutes, depending upon the parameters set for the run. The data can then be manipulated, labelled and/or time stamped using the interface 7.

### EXAMPLES

### Example 1

### Detection of Biocide Effect Upon Bacterial Growth in Nitrile Latex

### (a) Materials Used

non-sterile latex solution (test sample);
sterilised latex solution (reference sample); and
0.2% v/v Nipacide® (a parabens or 4-hydrobenzoic acid ester biocide;
obtained from Clariant UK Ltd, Horsforth, Calverley Lane, Horsforth, Leeds, West Yorkshire, LS18 4RP, UK).

### (b) Measurement Conditions

Incubation step: 300 seconds with constant stirring
Measuring cycle: 60 seconds
Measurements made: 2/second

### Temperature: 30° C.

### (c) Analysis of Efficacy of Biocide

1 ml of the test sample was added to a test receptacle having a headspace volume of approximately 3.4 ml and placed in the test chamber of a pressure sensing device as described herein. 1 ml of a reference sample was likewise added to a reference receptacle also having a headspace volume of approximately 3.4 ml which was then placed in the reference chamber of the same pressure sensing device.

Pressure measurement was then conducted as described herein according to the parameters defined in section (b) above. The biocide was then added to the test sample and the method was repeated immediately. The results of this analysis can be seen in FIG. 3 which demonstrates an immediate detection of pressure reduction. For example, a pressure reduction can be seen within just a few seconds and the pressure differential appears to be as sensitive as 1 Pa.

### Example 2

### Detection of Microbial Activity in Different Paint Samples

### (a) Materials Used

3 non-sterile paint solutions (test samples) with the following levels of microbial contamination (each sample having been assayed by conventional microbiological techniques):
(1) paint sample 1: 10⁷ cfu
(2) paint sample 2: 10² cfu
(3) paint sample 3: <10 cfu; and
   sterilised paint solution (reference sample)

### (b) Measurement Conditions

Incubation step: 300 seconds with constant stirring
Measuring cycle: 60 seconds
Measurements made: 2/second

### Temperature: 30° C.

### (c) Detection of Microbial Activity

1 g of a test sample was added to a test receptacle having a headspace volume of approximately 3.4 ml and placed in the test chamber of a pressure sensing device as described herein. 1 g of the reference sample was likewise added to a reference receptacle also having a headspace volume of approximately 3.4 ml which was then placed in the reference chamber of the same pressure sensing device.

Pressure measurement was then conducted as described herein according to the parameters defined in section (b) above. The method was then repeated with a different paint test sample. The results of this analysis can be seen in FIG. 4 which demonstrates a divergence in pressure difference between the three paint samples after just a few seconds, which corresponds to the level of contamination.

1. A method for determining the gas exchange of a test sample which comprises the steps of:
(a) adding a reference sample to a reference chamber:
(b) adding the test sample to a test chamber;
(c) sealing the reference chamber and test chamber from the external environment; and
(d) directly measuring the pressure differential between the reference chamber and test chamber over a specific time period.

2. A method as defined in claim 1, wherein the reference chamber and test chamber each comprise components capable of gas exchange.

3. A method as defined in claim 1, wherein said test sample has an unknown level of micro-organism activity and said reference sample has a known level of micro-organism activity.

4. A method as defined in claim 3, wherein said reference sample is sterile.

5. A method as defined in claim 1, wherein said test sample and said reference sample have equivalent levels of micro-organism activity.

6. A method as defined in claim 1, wherein each sample is introduced into each chamber in a receptacle.

7. A method as defined in claim 1, wherein one or more parameter adjustment steps are conducted prior to step (d).

8. A method as defined in claim 7, wherein the parameter adjustment step comprises one or more of temperature adjustment, pressure adjustment and mixing adjustment.

9. A method as defined in claim 7, wherein the parameter adjustment step is conducted by a processor.

10. A method as defined in claim 1, wherein at least one of pressure, temperature and mixing is controlled by processing means during step (d).

11. A method as defined in claim 1, wherein the measuring cycle is varied between 30 and 6000 seconds using the processor.

12. A method as defined in claim 1, wherein step (d) is repeated.

13. A method as defined in claim 1, wherein the pressure measurements are transferred to a processor and processed simultaneously with step (d).

14. A pressure sensing device comprising a plurality of sealable chambers and a direct pressure differential measurement sensor which is configured, in use, to directly measure the pressure differential between any of said chambers over a specific time period.

15. A pressure sensing device as defined in claim 14, wherein the plurality of sealable chambers comprise a test chamber and a reference chamber.

16. A pressure sensing device as defined in claim 14, wherein the plurality of sealable chambers additionally comprise a removable receptacle.

17. A pressure sensing device as defined in claim 16, wherein the receptacle is moulded substantially as a hollow cylinder, with a flat base and an open top having discrete vertical, upwardly extending protrusions.

18. A pressure sensing device as defined in claim 16, wherein the volume of the receptacle is in the range of 500 μl to 5000 μl,

19. A pressure sensing device as defined in claim 16, wherein the removable receptacle comprises a headspace marker.

20. A pressure sensing device as defined in claim 14, wherein each chamber additionally comprises a suitable CO₂ absorber.

21. A pressure sensing device as defined in claim 14, wherein each chamber additionally comprises an exhaust, and a connector from the valve to the chamber.

22. A pressure sensing device as defined in claim 21, wherein the exhaust comprise a gas-tight valve and a connector from the valve to the chamber.

23. A pressure sensing device as defined in claim 22, wherein the valve is a solenoid valve.

24. A pressure sensing device as defined in claim 14, wherein the direct pressure differential measurement sensor is an amplified pressure sensor.

25. A pressure sensing device as defined in claim 14, wherein the direct pressure differential measurement sensor is linked either directly or indirectly to a processor.

26. A pressure sensing device as defined in claim 25, wherein the processor is arranged to output the pressure differences between any of said chambers.

27. A pressure sensing device as defined in claim 14, which additionally comprises a temperature control.

28. A pressure sensing device as defined in claim 27, wherein the temperature control is a thermostatic enclosure.

29. A pressure sensing device as defined in claim 14, which additionally comprises a mixer.

30. A pressure sensing device as defined in claim 29, wherein the mixer is a magnetic stirrer.

31. A pressure sensing device as defined in claim 21, wherein at least one of the exhaust, temperature control and mixer are controlled by the processing means.

32. (canceled)

33. A micro-organism detection kit which comprises a device as defined in claim 14 and instructions to use said kit in accordance with a method as defined as follows:
(a) adding a reference sample to a reference chamber;
(b) adding the test sample to a test chamber;
(c) sealing the reference and test chambers from the external environment; and
(d) directly measuring the pressure differential between each of said chambers over a specific time period.

34. A method for monitoring biocide efficacy, said method comprising the use of a pressure sensing device comprising a plurality of sealable chambers and a direct pressure differential measurement sensor which is configured, in use, to directly measure the pressure differential between any of said chambers over a specific time period, said method further comprising the step of adding a biocide to one of said sealable chambers.
